(19) United States
(12) Patent Application Publication
(10) Pub. No.: US 2022/0293263 A1
(43) Pub. Date: Sep. 15, 2022

(54) SYSTEM, METHOD, AND APPARATUS FOR PROVIDING REMOTE HEALTHCARE

(71) Applicants: Marisa Sherry, New York, NY (US); Alon Israely, Seattle, WA (US)

(72) Inventors: Marisa Sherry, New York, NY (US); Alon Israely, Seattle, WA (US)

(21) Appl. No.: 17/687,326

(22) Filed: Mar. 4, 2022

Related U.S. Application Data
(60) Provisional application No. 63/156,422, filed on Mar. 4, 2021.

(51) Int. Cl. G16H 40/67

(52) U.S. Cl. CPC G16H 40/67

(57) ABSTRACT

The present disclosure generally relates to a system, method, and apparatus for providing remote healthcare. The system, method, and apparatus may include a patient monitoring device that may be used remotely by a patient and that may remotely communicate with the system. The system, method, and apparatus may also include a module and artificial intelligence for processing and utilizing data provided by the patient monitoring device and by an application of the system. The patient monitoring device may not provide measured information directly to the patient.

### RELATED APPLICATIONS

This application claims the benefit of U.S. Provisional Patent Application No. 63/156,422 filed Mar. 4, 2021, which is hereby incorporated by reference in its entirety.

### TECHNICAL FIELD

The present disclosure is directed to a system, method, and apparatus for providing healthcare, and more particularly, to a system, method, and apparatus for providing remote healthcare.

### BACKGROUND OF THE DISCLOSURE

Currently in the United States alone, an estimated eight million young people suffer from eating disorders such as anorexia and bulimia. These disorders are considered by experts to be the deadliest mental disorders of young people, with sufferers of these disorders having mortality rates ten times higher than their peers. An additional nine million people in the United States suffer from a lesser-known form of the disease called anorexia cachexia, which is triggered by certain cancers, chronic heart disease, kidney failure, and Alzheimer's. Patients with cancer and anorexia cachexia have an 80% mortality rate. An additional 20-30 million Americans engage in various degrees of disordered eating patterns that put them at risk either of falling into acute disease or relapsing into acute disease. An estimated 70% or more of individuals with these conditions never receive any treatment at all.

A foundational element for a successful recovery from these types of conditions is close monitoring of a patient's weight by a medical practitioner, but not by the patient. It is a well-established procedure among eating disorder specialists such as nutritionists and other related medical professionals that ensuring an individual patient has no access to their weight information is key to a successful treatment. For example, a patient having access to his or her own weight information may lead to detrimental results such as a relapse of the eating disorder, which may cause grave bodily injury or death. Further, keeping a patient from having access to his or her weight typically makes providing efficient treatment more difficult, as conventional treatment for limiting a patient's access to weight information typically involves in-person treatment utilizing a special weighing device that prevents a patient's access to weight information.

This physical logistical complication of conventional systems limits a clinician's ability to frequently monitor a patient's weight status and also hinders rapid response in certain emergency situations and also when working with patients and their primary medical providers to provide proactivity in treatment. These complications involved in conventional systems may be especially challenging when an eating disorder occurs in tandem with other medical diagnoses such as oncological issues.

The exemplary disclosed system, method, and apparatus of the present disclosure is directed to overcoming one or more of the shortcomings set forth above and/or other deficiencies in existing technology.

### BRIEF DESCRIPTION OF THE DRAWINGS

Accompanying this written specification is a collection of drawings of exemplary embodiments of the present disclosure. One of ordinary skill in the art would appreciate that these are merely exemplary embodiments, and additional and alternative embodiments may exist and still within the spirit of the disclosure as described herein.

FIG. 1A illustrates a top view of at least some exemplary embodiments of the present disclosure;

FIG. 1B illustrates a schematic view of at least some exemplary embodiments of the present disclosure;

FIG. 1C illustrates a schematic view of at least some exemplary embodiments of the present disclosure;

FIG. 1D illustrates a schematic view of at least some exemplary embodiments of the present disclosure;

FIG. 1E illustrates a schematic view of at least some exemplary embodiments of the present disclosure;

FIG. 2 illustrates a schematic view of at least some exemplary embodiments of the present disclosure;

FIG. 2A illustrates a detailed view of the schematic view of FIG. 2;

FIG. 2B illustrates a detailed view of the schematic view of FIG. 2;

FIG. 2C illustrates a detailed view of the schematic view of FIG. 2;

FIG. 3 illustrates a schematic view of at least some exemplary embodiments of the present disclosure;

FIG. 3A illustrates a detailed view of the schematic view of FIG. 3;

FIG. 3B illustrates a detailed view of the schematic view of FIG. 3;

FIG. 4A illustrates a schematic view of at least some exemplary embodiments of the present disclosure;

FIG. 4B illustrates a schematic view of at least some exemplary embodiments of the present disclosure;

FIG. 4C illustrates a schematic view of at least some exemplary embodiments of the present disclosure;

FIG. 4D illustrates a schematic view of at least some exemplary embodiments of the present disclosure;

FIG. 4E illustrates a schematic view of at least some exemplary embodiments of the present disclosure;

FIG. 4F illustrates a schematic view of at least some exemplary embodiments of the present disclosure;

FIG. 4G illustrates a schematic view of at least some exemplary embodiments of the present disclosure;

FIG. 4H illustrates a schematic view of at least some exemplary embodiments of the present disclosure;

FIG. 4I illustrates a schematic view of at least some exemplary embodiments of the present disclosure;

FIG. 4J illustrates a schematic view of at least some exemplary embodiments of the present disclosure;

FIG. 4K illustrates a schematic view of at least some exemplary embodiments of the present disclosure;

FIG. 4L illustrates a schematic view of at least some exemplary embodiments of the present disclosure;

FIG. 5 is a schematic illustration of an exemplary computing device, in accordance with at least some exemplary embodiments of the present disclosure;

FIG. 6 is a schematic illustration of an exemplary network, in accordance with at least some exemplary embodiments of the present disclosure; and

FIG. 7 is a schematic illustration of an exemplary network, in accordance with at least some exemplary embodiments of the present disclosure.

### DETAILED DESCRIPTION AND INDUSTRIAL APPLICABILITY

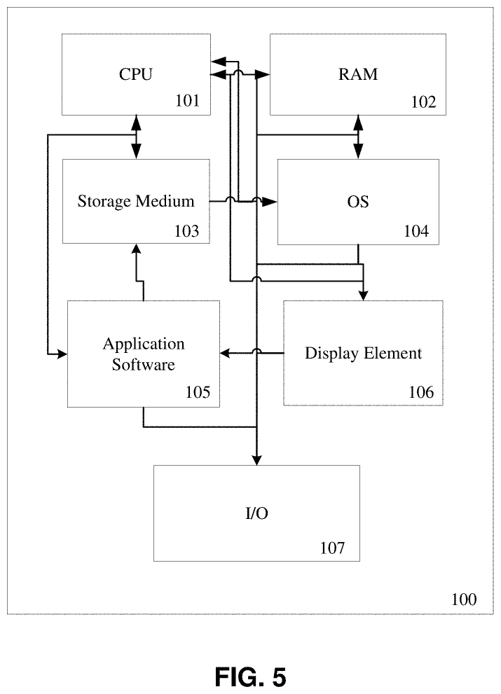

In at least some exemplary embodiments, the exemplary disclosed system, method, and apparatus may be a system, method, and apparatus for providing remote healthcare. For example, the exemplary disclosed system, method, and apparatus may be a remote weight measuring system including for example a numberless scale (e.g., weightless scale) and information service. The exemplary disclosed system, method, and apparatus may operate using computing systems and networks similar to the exemplary systems and networks described below regarding FIGS. 5, 6, and 7.

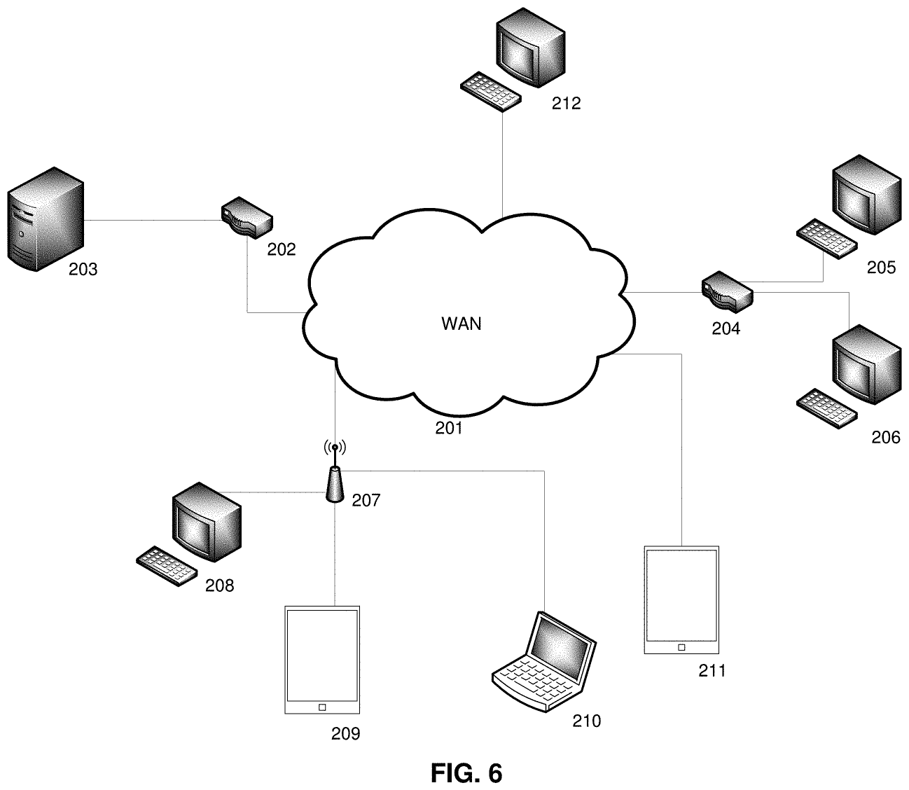

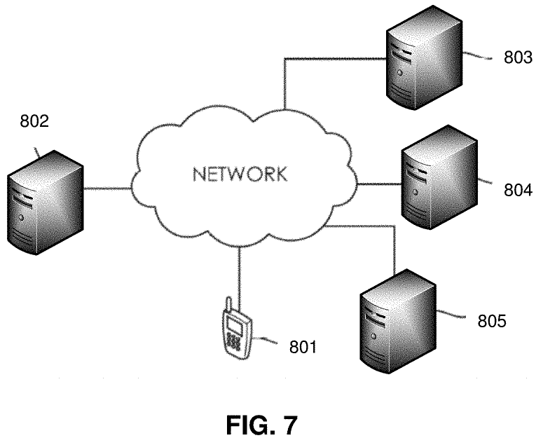

In at least some exemplary embodiments, the exemplary disclosed system, method, and apparatus may be a remote weight measuring system including a measurement device such as a physical weight measuring device adapted with no local measurement readout and attached to a remotely accessible readout via a software mechanism. The exemplary disclosed measurement device may measure a bodily attribute of a user such as, for example, a bodily attribute data value. The remote weight measuring system may include a platform that supports the weight of a human body, a set of scales to measure the weight input from the platform, and an electronic board to process the information received from the scale. The remote weight measuring system may also include software to further process the information, transfer and store the information, and display the information as a digital readout on an independent device such as a smartphone or personal computer (e.g., a device of a medical practitioner such as a clinician). A user may place himself or herself on a physical platform weighing device, wherein the weight information is stored (e.g., but not displayed) and transferred according to software instructions (e.g., via communication systems for example as illustrated in FIGS. 6 and 7). A user such as a clinician may receive that information for medical clinical purposes. The clinician may be any professional or worker who provides healthcare services to a user. The capture, storage, processing, and transfer of information may be managed to be compliant with HIPAA and other U.S. and international health related digital laws and regulations (e.g., 42 U.S. Code § 1320d-2).

The exemplary disclosed system, method, and apparatus may include a cloud-based platform for remote patient monitoring of eating disorders that makes it possible for clinicians to monitor the health of thousands of patients or more. The exemplary disclosed system, method, and apparatus may alert clinicians to small changes in patient weight that may indicate a worsening of a condition such as an eating disorder, a relapse into acute disease, or specific binge and purge episodes that may be dangerous and lead to a health crisis for the patient.

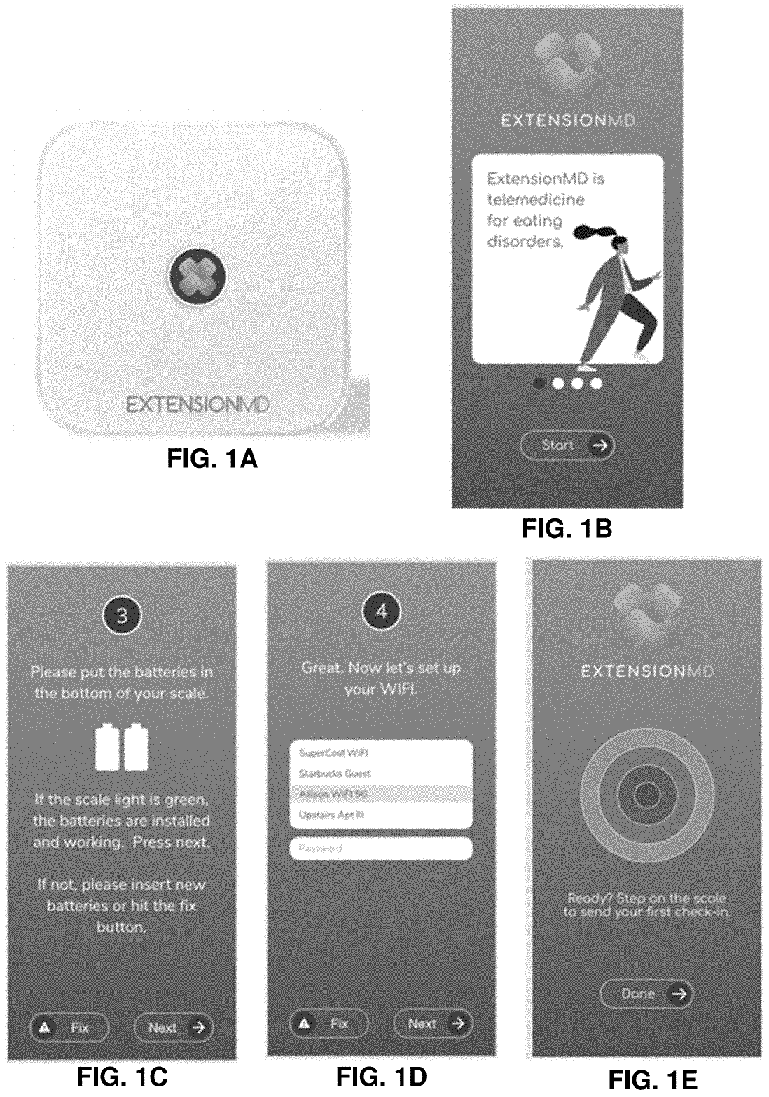

The exemplary disclosed system, method, and apparatus may include a weight scale (e.g., a bodyweight scale) such as a numberless scale (e.g., as illustrated in FIG. 1) that may be connected by any suitable communication technique (e.g., such as described below regarding FIGS. 6 and 7, e.g., Wifi-connected) and that sends weight data from patients' homes directly to clinicians without the patient seeing a number or being triggered to engage in negative behaviors based on seeing their weight displayed. Patients may simply step on and off a scale at any location (e.g., at home), which may be performed such that the patient does not see the weight value.

The exemplary disclosed system, method, and apparatus may include a software application or API (e.g., a patient app that may operate on any desired patient device such as the devices described below regarding FIGS. 5-7) that may instruct patients when to weigh themselves (e.g., which may be set by clinicians) and that may offer intuitively simple food and mood logs. The application may include any suitable modules or other components for example as illustrated in FIGS. 5-7. The application (app) may connect to third-party “IoT” devices such as Apple Watch and Fitbit devices to provide a fuller picture of patient behavior, which clinicians may use as part of treatment.

The exemplary disclosed system, method, and apparatus may provide a cloud-based platform that may assist clinicians in providing healthcare. Some exemplary ways the exemplary disclosed system may assist clinicians include making it easier for clinicians to remotely monitor patients and scale telehealth from dozens of patients to millions, allowing clinicians to customize alert triggers for each patient, taking into account their disease history and trajectory, charting a patient's success (e.g., over days, weeks, and/or years) for use by a clinician, securely sharing data with an entire clinical team with substantially full HIPAA compliance (e.g., the exemplary disclosed scale may send fully encrypted data to system servers and the system may manage HIPAA permissions from patients), and including artificial intelligence (AI) for example as described below that identifies patterns of disorderly eating before they become critical and that provides advance warning to clinicians and the treatment team.

The exemplary disclosed system, method, and apparatus may include a platform to connect eating disorder patients with their clinical teams and help clinicians expand their telemedicine practice. The exemplary disclosed system, method, and apparatus may include an artificial intelligence (AI) engine that receives (e.g., ingests or listens to) millions of health data (e.g., signals) transported over the system network (e.g., data of weights, food, mood, sleep, exercise, self-reported compulsive thoughts and disordered eating behaviors) and learns to identify the early signs of patient distress and patient improvement that may be used to direct patient treatment. The exemplary disclosed system, method, and apparatus may identify a patient's propensity to purge or to restrict, and may identify a potential avenue to help a patient reduce compulsive thoughts around food. The exemplary disclosed AI engine of the exemplary disclosed system may be part of a secure cloud platform for clinicians that is intended to help guide patient treatment and that can scale (e.g., scale quickly) across both a number of clinicians and a number of patients. Ultimately, millions of acute patients and people at risk of the disorder may receive monitoring services at a fraction of the current cost of care. The exemplary disclosed system, method, and apparatus may be used to treat a majority of at-risk patients using an app that may guide patients to improve behaviors and reduce compulsive thoughts while the exemplary disclosed numberless scale (e.g., does not display data) and AI may monitor patient progress for signs of trouble.

Relatively higher levels of care (e.g., typically virtually with facetime or similar systems or in real life) with clinicians may be suitable for some patients. The exemplary disclosed system, method, and apparatus may include AI that may seek to identify early warning signs and guide those patients to higher levels of care that may be provided from a network of telehealth professionals (e.g., dieticians, physicians, and mental health workers) of the exemplary disclosed system. The exemplary disclosed system, method, and apparatus may be used to provide monitoring and accountability telehealth services to eating disorder patients who are considered to be in recovery (e.g., discharged from care and on their own).

A user (e.g., a client) may have a patient monitoring device located for example in a residence, workplace, or other desired location where a user may utilize the patient monitoring device as appropriate (e.g., as part of patient treatment by a clinician, such as remote treatment). The patient monitoring device may for example be a device as illustrated in FIG. 1A. The exemplary disclosed patient monitoring device may be a scale for measuring weight. The exemplary disclosed patient monitoring device may provide little or substantially no (e.g., very little) feedback or data to users. The exemplary disclosed patient monitoring device may include a colored light that indicates when the device is ready for use, when a measurement has been made by the device (e.g., a reading has been recorded), when that reading has been sent to a provider (e.g., a clinician or healthcare practitioner treating the user), and/or whether an error has occurred with the device. The exemplary disclosed patient monitoring device may remotely connect (e.g., via any suitable communication technique for example as described herein, e.g., via WiFi) to a network (e.g., a backend of a service provided by the exemplary disclosed system), to securely transmit weight values, date/time information, data related to the device unique identifier, device management data, and/or any other suitable data.

The exemplary disclosed patient monitoring device may include a light indicator for example as illustrated in FIG. 1A that operates to indicate various states or modes of operation of the device. For example, the light indicator may emit a first colored light (e.g., magenta) that may indicate that the device is waiting to communicate (e.g., connect to WiFi). The light indicator may emit a second colored light (e.g., white) that may indicate that the device is ready for use (e.g., that the device such as a scale is ready for use and that a user should step on the scale). The light indicator may emit a third colored light (e.g., blue) that may indicate that the user should hold still until the device (e.g., scale) obtains a stable reading. The light indicator may emit a fourth colored light (e.g., green) that may indicate that a measurement has been taken (e.g., that a reading has been acquired and that a user should step off the device and/or if a user does not see a green light, the user should step off the device for a few seconds and then step back on). The light indicator may emit a fifth colored light (e.g., yellow) that may indicate that data is being sent (e.g., to a clinician). The light indicator may emit a sixth colored light (e.g., three lights such as three blue lights) that may indicate that the device is powering down (e.g., going to sleep).

The exemplary disclosed patient monitoring device for example as illustrated in FIG. 1A may calculate body fat and body water levels using bioelectric impedance analysis (BIA). The exemplary disclosed device may include a display (e.g., light display) disposed on a floor and/or wall. The exemplary disclosed device may vibrate when a measurement has been taken (e.g., weight has been measured or registered). The exemplary disclosed device may indicate (via a display and/or audio instructions) to a user when to step on and off the device (e.g., the scale) and whether batteries are running low. The exemplary disclosed device may be substantially waterproof or water-resistant. The exemplary disclosed device may provide encrypted (e.g., substantially fully encrypted) data transmission via any suitable communication technique for example as described herein (e.g., to cloud servers).

The exemplary disclosed system, method, and apparatus may include components similar to those described at FIGS. 5-7 (e.g., a backend of the system) that include web-services, business logic, databases, AI engine, and related IT infrastructure that may all be designed using dual concepts of security- and privacy-by design and implementing modern security and data protection practices. Such a configuration may provide for data collection, processing, and storage that meets or exceeds the requirements of HIPAA, e.g., including reliance on HI-TECH, accepted standards (such as NIST 800-53), documented risk assessments, and all patient notification and request-for-information criteria. The exemplary disclosed device (e.g., as illustrated in FIG. 1) may communicate with the backend using secure communications that may be protected by encryption (e.g., TLS 1.1+). PHI may be sent between the exemplary disclosed device and the backend of the system. Weight telemetry and GUIDS (machine readable identifiers) may be sent between the exemplary disclosed device and the backend of the system.

The exemplary disclosed system, method, and apparatus may include an application that may be downloaded (e.g., on a smartphone, tablet, or any other computing device for example as illustrated in FIGS. 5-7) for example as illustrated in FIG. 1B. A user may then be instructed to configure (e.g., insert batteries) the exemplary disclosed device (e.g., for example as illustrated in FIG. 1A) for example as shown in FIG. 1C. For example, the batteries or other power sources may be correctly installed if the indicator of the exemplary disclosed device emits a predetermined color (e.g., green). Otherwise, new batteries may be inserted or a control feature (e.g., “fix” button) of the exemplary disclosed device may be activated.

As illustrated in FIG. 1D, communication may be set up on the exemplary disclosed device (e.g., WiFi set up). A user may choose a proper network SSID and input a password/phrase so that the exemplary disclosed device for example as illustrated in FIG. 1A (e.g., a scale) may automatically connect to a selected network. For example, using the network's Internet gateway (e.g., for example as illustrated in FIGS. 5-7), the app may communicate with the backend of the system using an encrypted transmission mode. If an error occurs, the exemplary disclosed app may automatically re-attempt to connect to the selected SSID, and may eventually time out and display an error message and/or instructions for contacting technical support.

As illustrated in FIG. 1E, the exemplary disclosed system, method, and apparatus may perform a first “Check-In” (e.g., the term “weigh-in” may be avoided so as to avoid triggering a patient). Data such as weight data and related information may be communicated, using for example HTTPS over the Internet to the system backend. The transferred data may be stored in a database along with device identification information to allow for Check-In or weigh-in data to be joined to other patient information and presented to the clinician via a secure web-browser. If data is not successfully transmitted from the exemplary disclosed device (for example as illustrated in FIG. 1A) to the backend, errors may be captured and depending upon the error type (e.g., no Internet, bad weigh-in reading), instructions may be presented to the user. Also, the exemplary disclosed system may log the error and may attempt to recover.

The exemplary disclosed system, method, and apparatus may perform various operations following the above exemplary disclosed setup steps. The exemplary disclosed system may alert users when their respective clinician requests them to perform a check-in. Check-ins may be scheduled or on-the-fly (e.g., “flash check-ins” to reduce cheating). The exemplary disclosed system may provide daily (e.g., or at any other desired interval) affirmations to encourage users to stay on track and within their program parameters. The exemplary disclosed system may record answers from patients to standardized mental health questions that may help determine levels of self-reported disordered eating. The exemplary disclosed system may record mood information as reported by a patient via the patient's interaction with the exemplary disclosed app (e.g. a mood palate) that may convert the patient inputs to data that may be used by the exemplary disclosed AI engine. The exemplary disclosed system may record food consumption as reported by a patient, which may also be converted to data used by the exemplary disclosed AI engine. The exemplary disclosed system may record health signals from other IoT devices such as Apple Watch, Samsung Watch, Fitbit and other suitable device (e.g., newer, more specialized devices). Such data (e.g., signals) may include sleep patterns, exercise/movement, and heart rate information (e.g., and/or capture other bio telemetry in a non-invasive format). The above data may be used (e.g., processed) in connection with measured data provided by the exemplary device for example as illustrated in FIG. 1A (e.g., weight data), and used by the exemplary disclosed AI engine to offer predictions, treatment plan changes, and other informative health data. The exemplary disclosed system may allow patients to add and remove clinicians from their team with HIPAA compliance (e.g., substantially full compliance). The exemplary disclosed system may send and receive in-app chats and messages from clinicians securely and with HIPAA compliance (e.g., substantially full compliance).

The exemplary disclosed system, method, and apparatus may provide for weight Check-Ins. Patients may be directed by messages in the app and may push messages sent to their phone to perform a Check-In. A Check-In may include using the exemplary disclosed device for example as illustrated in FIG. 1A (e.g., stepping on the scale) and sending a reading to a clinician. A clinician, using a Clinician Cloud platform for example including components described in FIGS. 5-7, may both schedule Check-Ins at regular intervals or request an on-the-fly, ad-hoc “flash Check-In” that may serve to reduce cheating. Such Check-Ins may provide a check against patients working around (e.g., cheating or “gaming”) the highly scheduled in-person Check-In or weigh-in process (e.g., which may be a challenge in treating eating-disorders). Users may step on the scale without a request from their clinician. That data may still enter their patient record, but such activity may not be encouraged by the system as it may worsen a patient's compulsion to check their weight. If the exemplary disclosed system detects an excessive amount of non-scheduled Check-Ins, the exemplary disclosed device for example as illustrated in FIG. 1A may shut down or emit an alert (e.g., blink red) and/or may send an alert to the treating clinician (e.g., transfer alert data via communication techniques for example as described herein).

The exemplary disclosed system, method, and apparatus may provide information and data including daily affirmations and encouragement to users. A relatively small amount of highly encouraging information may be provided to patients, which may help patients feel confident when they are staying on a treatment plan (e.g., without over-signaling and encouraging obsessive thoughts). A patient may receive affirmation data (e.g., at any desired constant or variable interval such as daily) to help the patient remain on the path to recovery (e.g., by adhering to a treatment plan). This affirmation data may be based on each patient's individual goals. Such affirmation data may be configured based on input from the patient, input from the clinician, and/or based on a predetermined operation of the exemplary disclosed system.

The exemplary disclosed system, method, and apparatus may store (e.g., record) data of patient answers to medical health questions. For example, any suitable eating disorder diagnostic tests may be used (e.g., that utilize self-reporting). The exemplary disclosed AI may configure combinations of questions and may determine optimal times to ask questions and an amount of questions to ask at one time.

The exemplary disclosed system, method, and apparatus may record and store mood data. At any time (e.g., via the exemplary disclosed app), users may enter input data indicative of their current mood. The exemplary disclosed system may also prompt users to enter mood input data at any desired constant or variable interval. For example, users may be able to choose their current mood (e.g., sad, mad, fearful, or joyful) and rate the mood intensity (e.g., from 1-5) for example via the app. This exemplary disclosed input and interaction design (e.g., part of the app interface) may provide for data transfer to the exemplary disclosed AI engine, which may use the data to offer treatment advice.

The exemplary disclosed system, method, and apparatus may record and store food data. At any time (e.g., via the exemplary disclosed app), users may enter input data indicative of meals, snacks, and beverages that they consume. Users may enter data manually by entering text, search and choose from a list of prebuilt foods of the system's database, and/or take a photo or video of the food consumed. The system may employ visual recognition techniques to correctly identify both a type and a quantity of food based on picture or video data. Although the system may allow users to enter portion sizes, the system may also present food data in a format to minimize any food obsession (e.g., by not offering significant details of the food). The exemplary system may not provide calorie count data to users, to provide for eating disorder patients to eat regularly, free of mental distress and compulsive thoughts.

The exemplary disclosed system, method, and apparatus may record and store third party data (e.g., signals). The exemplary disclosed app (e.g., user device) may connect with third-party devices and other sources of health information to construct a fuller picture of a patient's health and risks. The exemplary disclosed app (e.g., user device) may utilize standard APIs to connect with devices such as Apple Watch, Samsung Watch, FitBit, third-party activity apps such as RunKeeper and other work-out apps, diet trackers like MyFitnessPal, and/or any other suitable devices and apps.

The exemplary disclosed system, method, and apparatus may record and store any suitable data (e.g., signals). The exemplary disclosed data may collect data (e.g., signals) from a user to attempt to determine (e.g., via an operation of the exemplary disclosed AI) a severity of a user's disease, their current state, and/or their propensity to worsen or improve. The collected data (e.g., signals) may include weights from the exemplary disclosed device (e.g., scale) for example as illustrated in FIG. 1A, differences in weight data between Check-Ins, frequency of missed weight Check-Ins, self-reported (e.g., by a user via the exemplary disclosed app) disordered eating behavior data such as restricting, binging, purging or engaging in thoughts such as excessive self judgement, self-reported mood, self-reported food intake, and/or data from third party IoT devices such as Apple Watch and Fitbit (e.g., which may record heart rate, exercise, movement, sleep patterns and other health data or signals). Signals and data (e.g., data or telemetry from the scale, third-party devices, and data inputs from the user such as a patient) may be used by the exemplary disclosed AI engine. The exemplary disclosed AI engine may include machine learning algorithms and expert/business logic for learning to identify early signs of patient distress and patient improvement, which together with workflows of the exemplary disclosed system described herein, may be used to direct patient treatment. The data and signals may provide data (e.g., raw data) for the exemplary disclosed system to use to analyze a patient's propensity for certain activities related to a healthcare condition (e.g., eating disorders such as when a patient may purge or restrict food intake). The exemplary disclosed data and signals may be used to identify techniques for reducing compulsive thoughts involving food. Treatment may thereby be driven using the exemplary disclosed device for example as illustrated in FIG. 1A, the exemplary disclosed app, and the exemplary disclosed backend of the system.

The exemplary disclosed system, method, and apparatus may provide for adding and removing clinicians to allow users (e.g., patients) to be in full control of their care. If a user such as a patient is working with a clinical team, that user may invite (e.g., via the exemplary disclosed app) clinicians to join a program provided by the exemplary disclosed system for monitoring progress by users (e.g., patients). Using the exemplary disclosed system, clinicians may add team members (e.g., with explicit permission of the user such as the patient). The user may have access to a network of clinicians via the exemplary disclosed system. The exemplary disclosed system (e.g., the exemplary disclosed AI) may recommend treatment steps and actions to a patient based on the data collected and processed by the system. Also for example, a user (e.g., a patient) may remove a clinician from their care team.

The exemplary disclosed system, method, and apparatus may operate in a substantially fully HIPAA compliant manner. Patients may e-sign authorization for the exemplary disclosed system to share data with clinicians. Patient data may be shared when the patient explicitly approves. Before any treatment team members recommended by other clinicians via the exemplary disclosed system are added to the patient's team, the patient may be requested to approve the change. Clinicians may sign (e.g., all clinicians sign) standard HIPAA terms and conditions that outline proper use of personal health information, notice criteria, and acknowledgements (e.g., including having proper business associate agreements in-place when suitable).

The exemplary disclosed system, method, and apparatus may provide for secure communication between users (e.g., patients) and clinicians. The exemplary disclosed system may provide secure, private communication directly within the exemplary disclosed app. For example, the exemplary disclosed app interface may provide a private chat channel. Information of that communication (e.g., private chat) may be added as data to a patient's electronic medical record.

The exemplary disclosed app may provide a food log, exercise log, sleep log, and/or mood log. The exemplary disclosed app may set daily or weekly (e.g., or any other interval) goals and check a user's progress against those goals. The exemplary disclosed app may connect to other iOT health devices such as FitBit and Apple Watch and collect their data. The exemplary disclosed app may provide daily affirmations (e.g., or at any other desired intervals) to encourage and guide a user's recovery. The exemplary disclosed app may allow for a user to add or delete members of that user's clinical team. The exemplary disclosed app may provide real-time or near real-time chat messaging with a clinical team, which may be substantially fully HIPAA compliant. The exemplary disclosed app may allow a user to manage a user account (e.g., including profile, billing, and insurance). The exemplary disclosed app may receive weight Check-In requests (e.g., scheduled and flashed) from a clinical team and notify the user of those requests. The exemplary disclosed app may receive recommendations from the exemplary disclosed system (e.g., the exemplary disclosed AI) regarding the user's level of care. The exemplary disclosed app may provide for live video telemedicine Check-Ins with a user's doctors and clinical team.

The exemplary disclosed app (e.g., and/or a clinician interface that may operate similarly to the exemplary disclosed app) may provide for remote monitoring of patients (e.g., including a patient's weight, food intake, moods, exercise amounts, sleep, recovery goals, recovery goal progress, and/or other physical and emotional signals). The exemplary disclosed app (e.g., and/or clinician interface) may provide for inviting patients to be remotely monitored, securely sharing data with a patient's clinical team, and/or providing secure real-time or near real-time chat (e.g., text, pictures, and/or video) between a patient and clinical team members (e.g., or between clinician team members). The exemplary disclosed app (e.g., and/or clinician interface) may provide for conducting audio/video synchronous and asynchronous telehealth sessions with patients. The exemplary disclosed app (e.g., and/or clinician interface) may provide for transferring and receiving propensity scores determined by the exemplary disclosed AI to help guide recovery treatment and patient risk analysis. The exemplary disclosed app (e.g., and/or clinician interface) may provide for setting a critical weight change number (e.g., a weight change delta that triggers an alert to the clinician), for example based on an operation of the exemplary disclosed AI. The exemplary disclosed app (e.g., and/or clinician interface) may provide patient reports that may include critical metrics (e.g., weight, mood, and food) that may change over time (e.g., daily, weekly, monthly, yearly, or any other desired time period.). The exemplary disclosed app (e.g., and/or clinician interface) may provide for determining Body Mass Index (e.g., based on an operation of the exemplary disclosed device for example as illustrated in FIG. 1A and the exemplary disclosed system). The exemplary disclosed app (e.g., and/or clinician interface) may provide for entering a patient's height (e.g., based on receiving input from a clinician team member or the user).

The exemplary disclosed AI may ingest some or substantially all patient data (e.g., signals) collected via any of the client app, the exemplary disclosed device for example as illustrated in FIG. 1A, and/or third-party apps and devices. The exemplary disclosed AI may ingest clinician assessments of patients' state, risk level, and prognosis. The exemplary disclosed AI may be a building intelligence that operates to understand patterns of disordered eating. The exemplary disclosed AI may create propensity scores that describe patients' current disease state, risk level, and trajectory (e.g., such as propensity to binge, propensity to purge, propensity to need higher or lower level of care, propensity to relapse, propensity to stay stable, propensity to have a crisis, and/or propensity to be in recovery). The exemplary disclosed AI may create propensity scores that may be shown to clinicians (e.g., both inside and outside of a clinician network of the exemplary disclosed system) to assist the clinicians in assessing patient risk and making treatment recommendations. The exemplary disclosed AI may use machine learning based on past and current patients to assess a risk level of undiagnosed users.

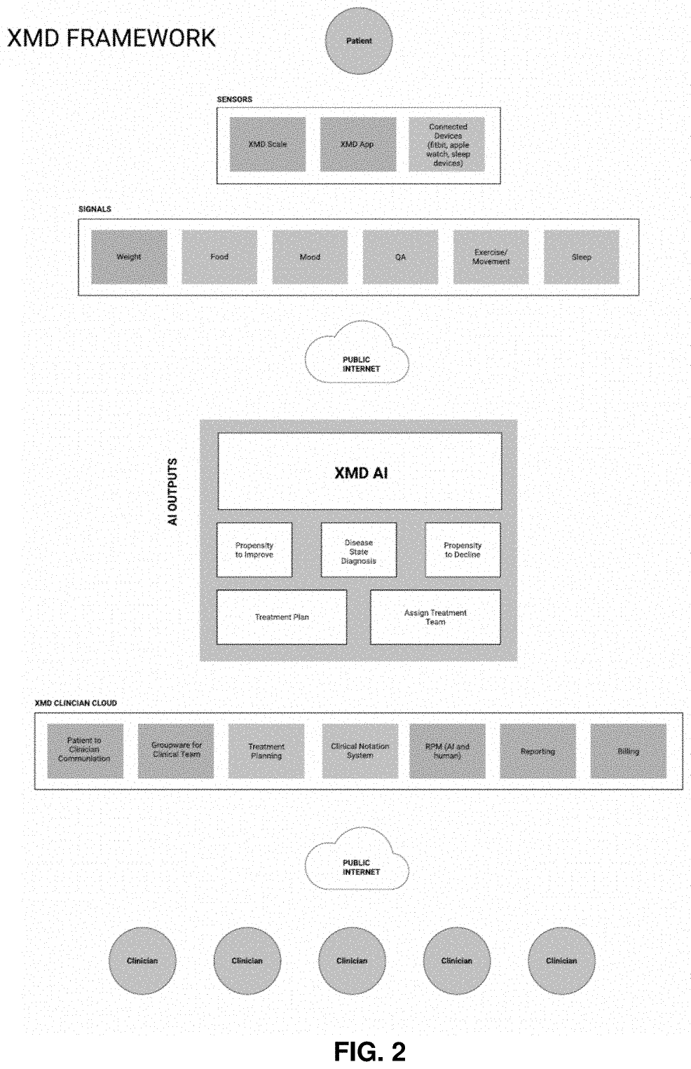

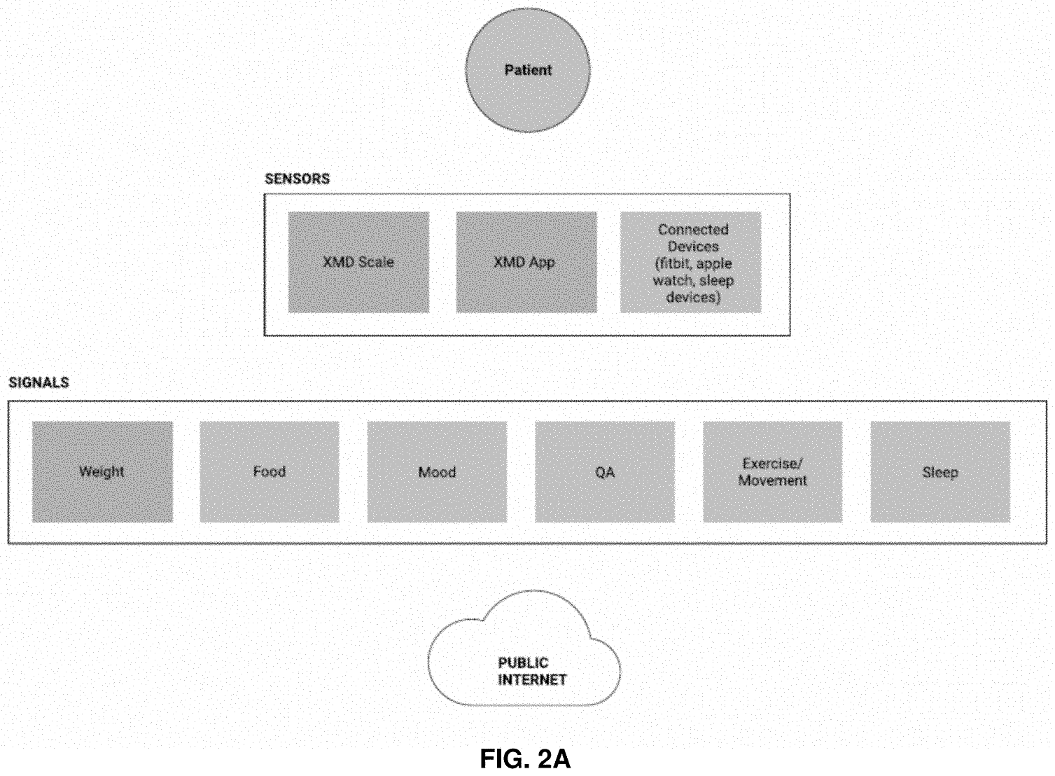

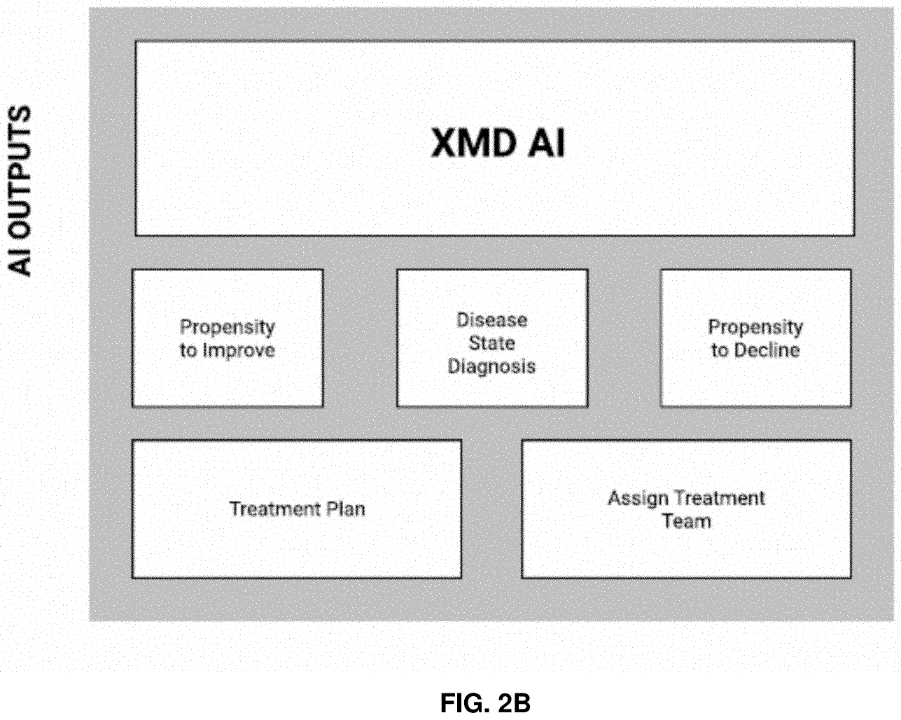

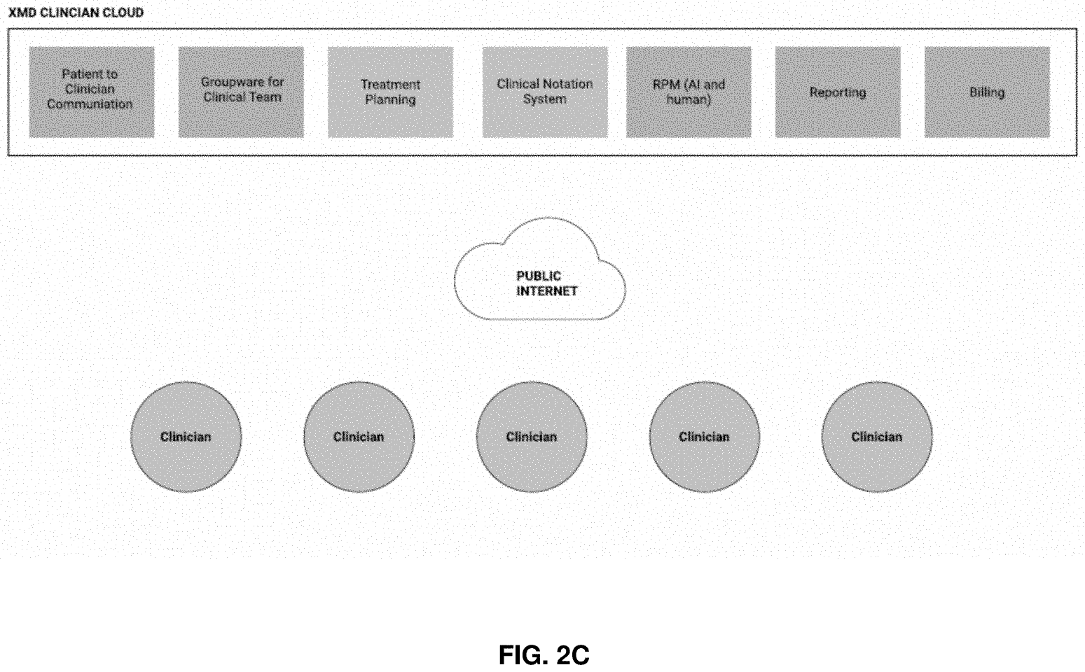

FIGS. 2, 2A, 2B, and 2C illustrate an exemplary disclosed framework (e.g., architecture) for the exemplary disclosed system.

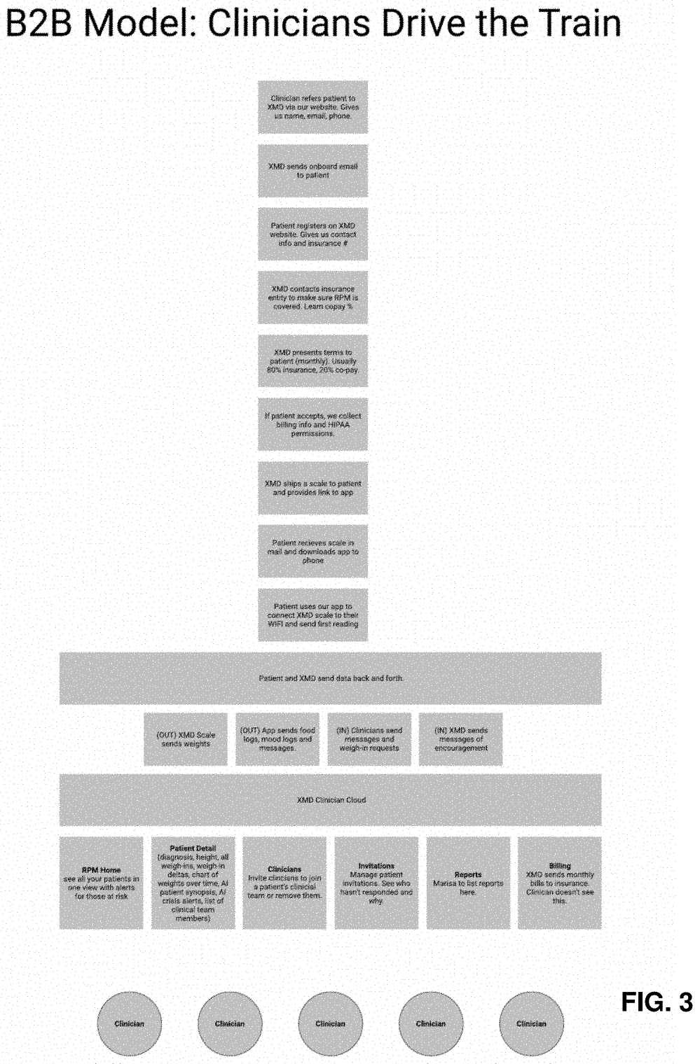

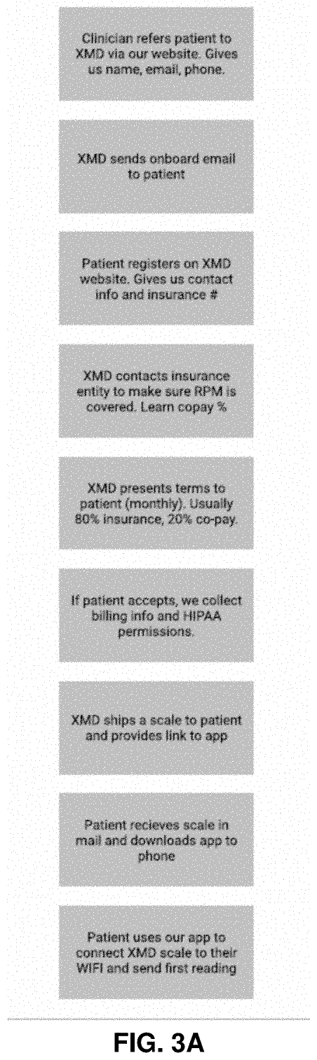

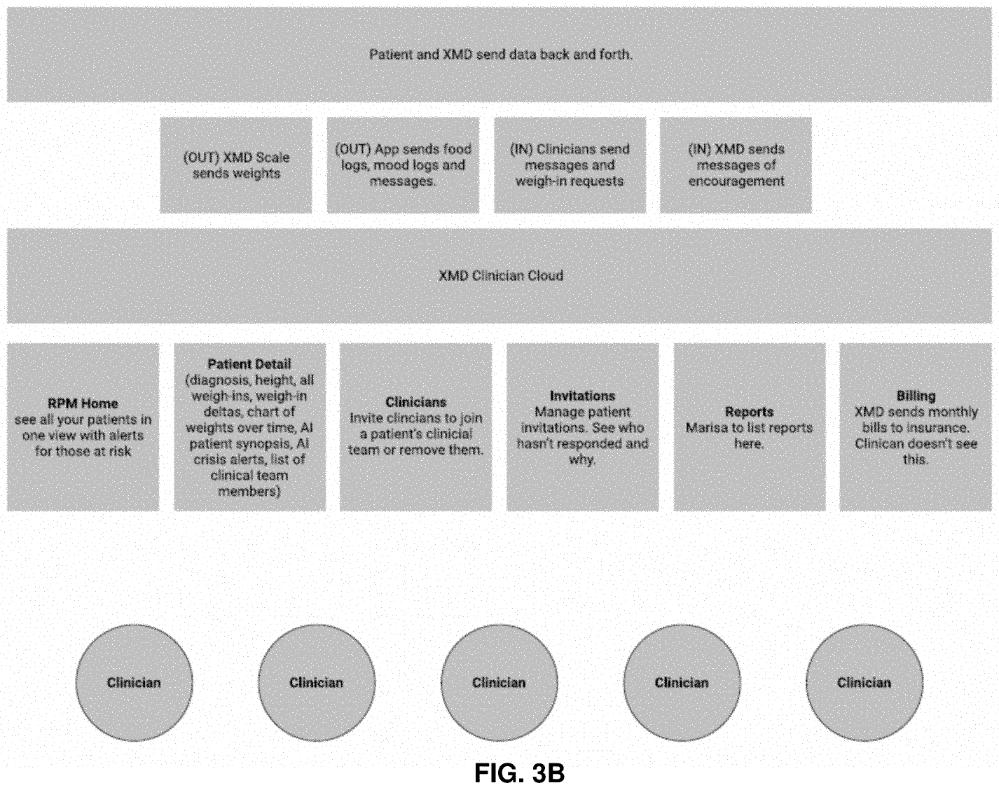

FIGS. 3, 3A, and 3B illustrate an exemplary disclosed model and process steps for performing the exemplary disclosed method.

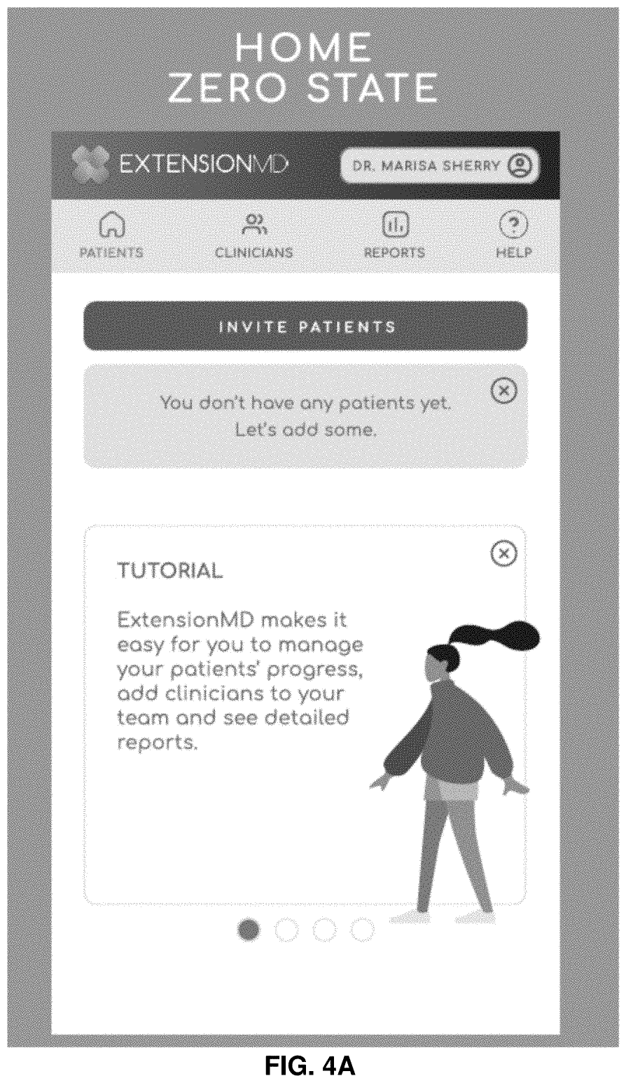

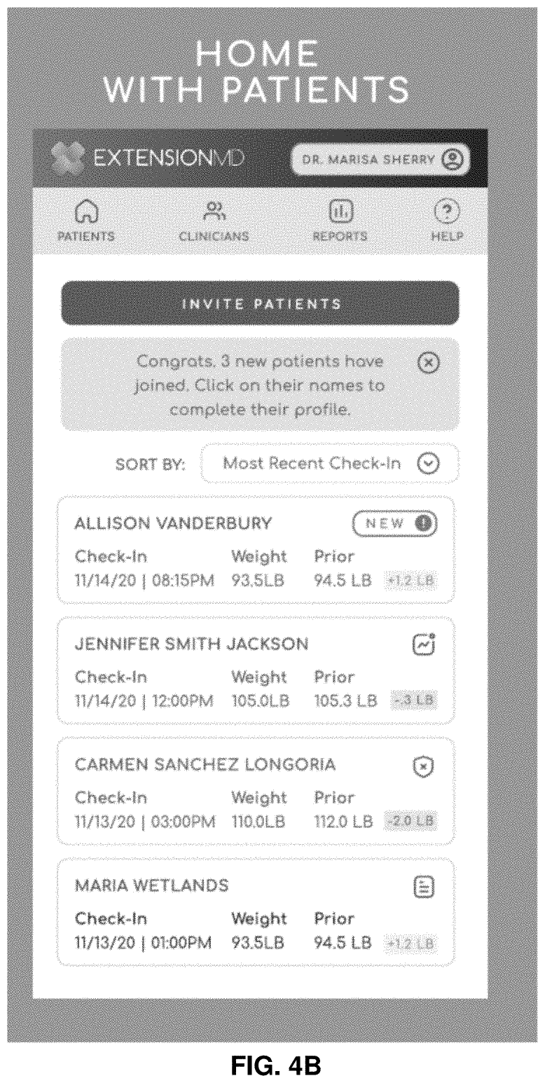

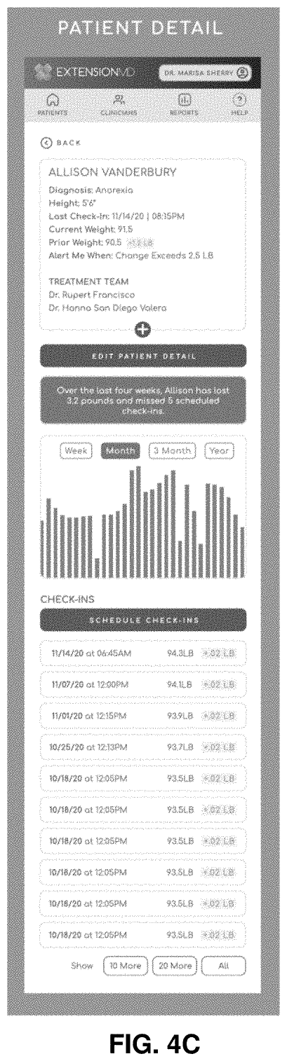

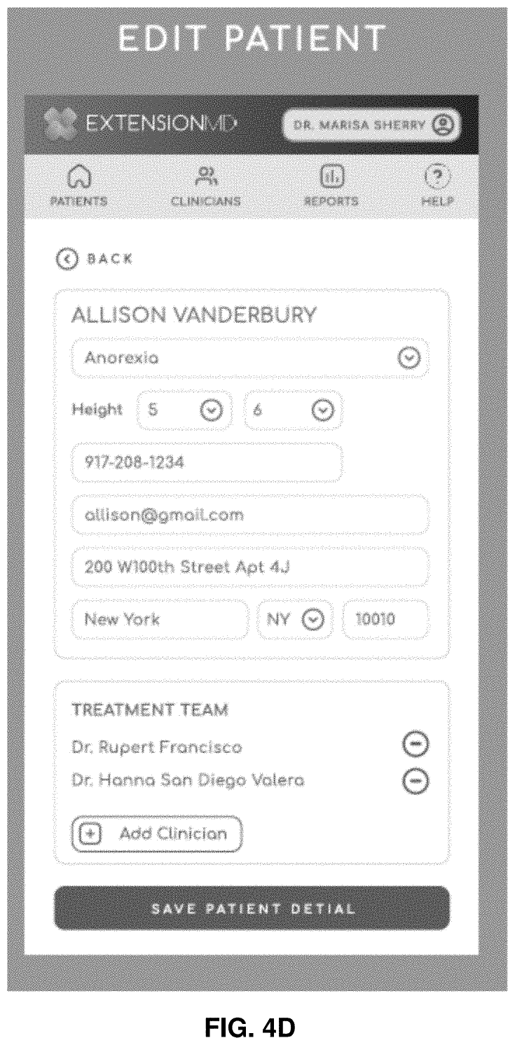

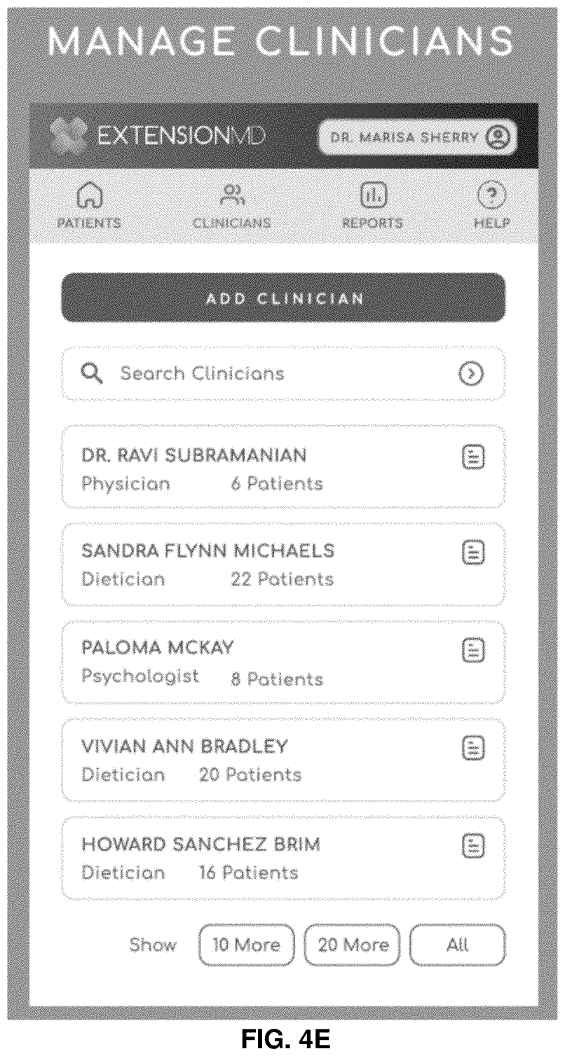

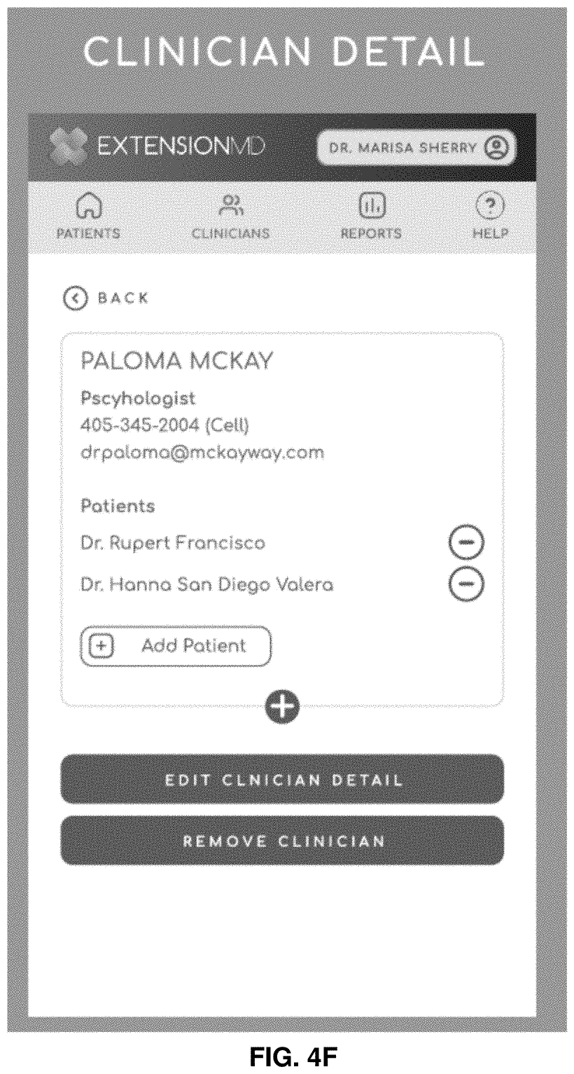

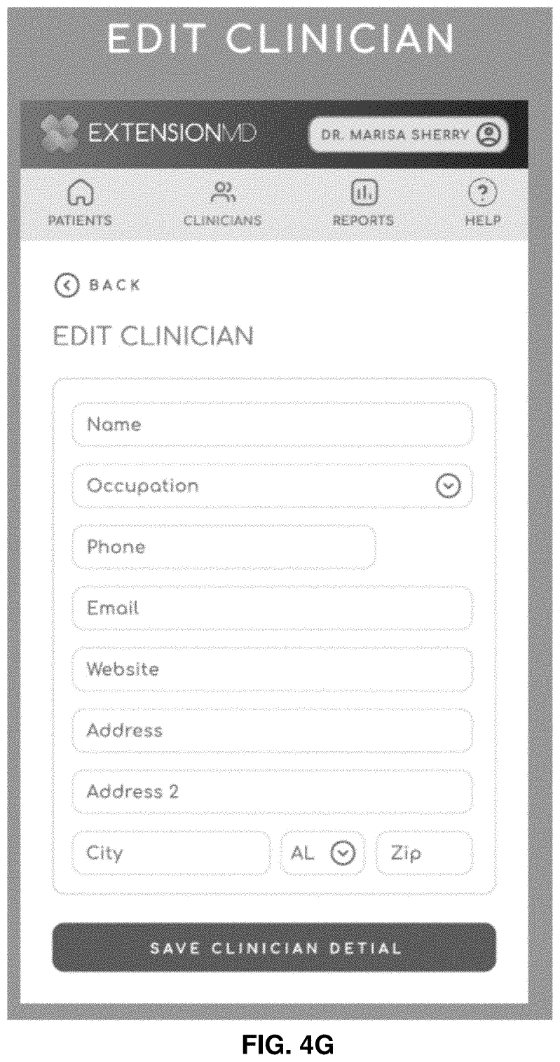

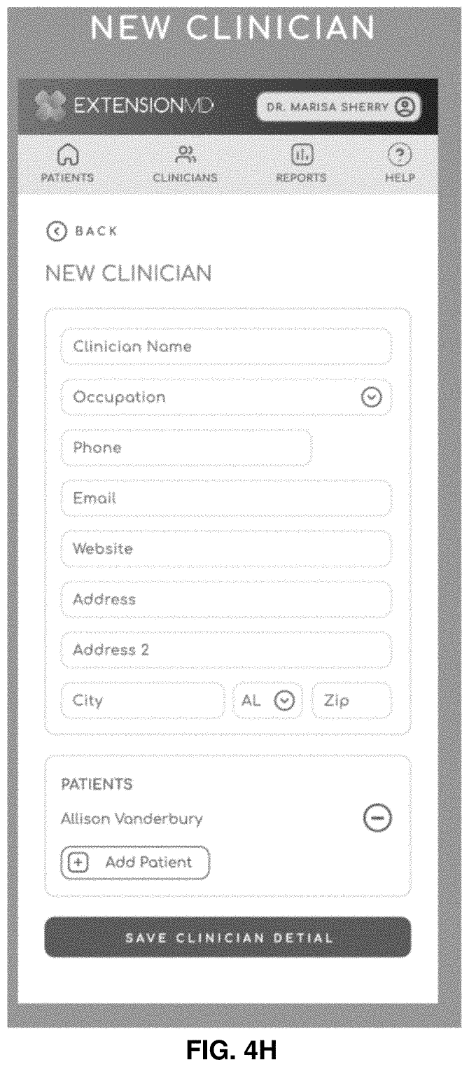

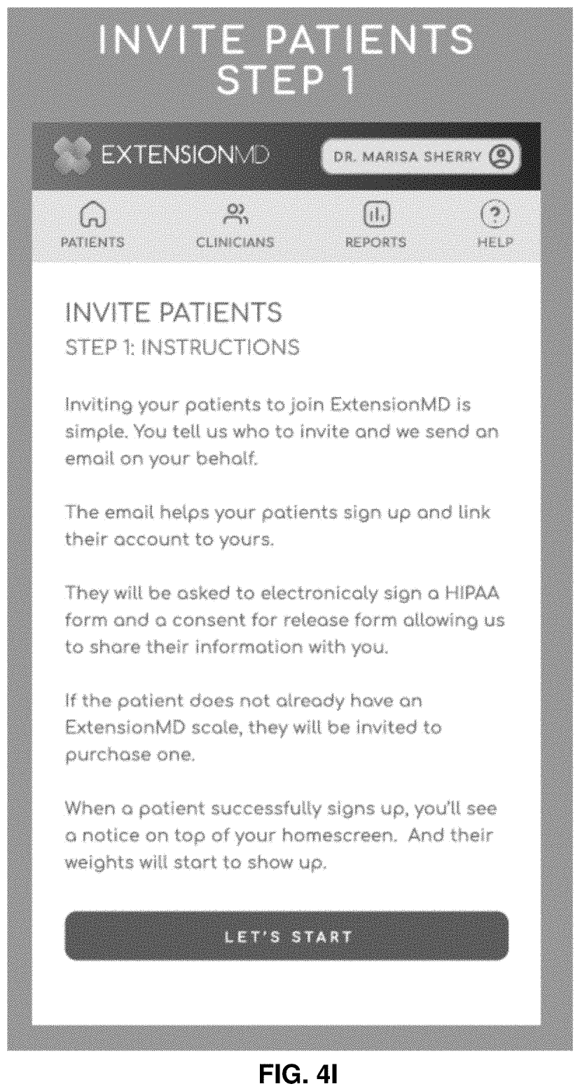

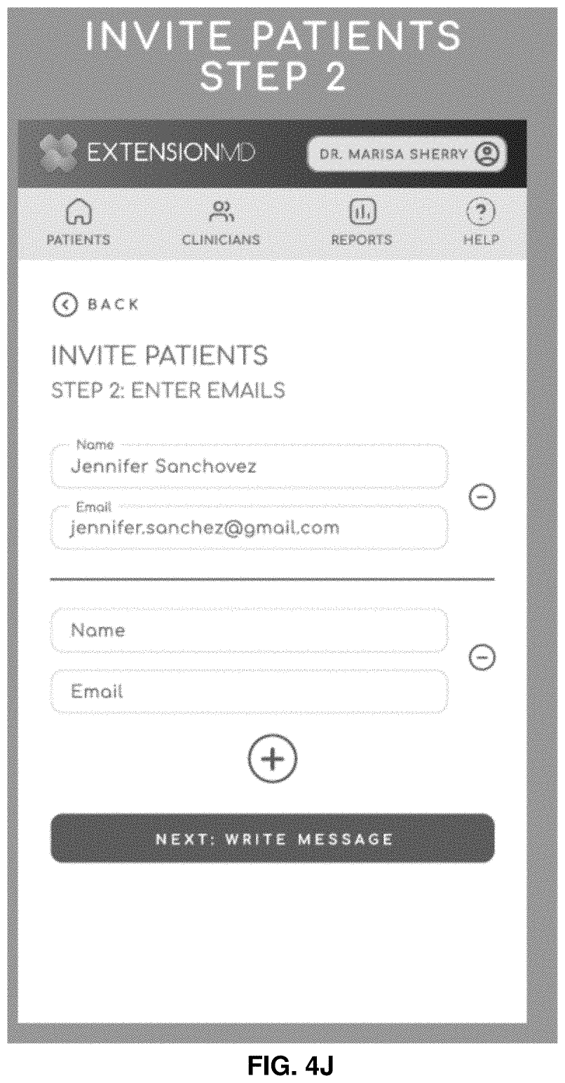

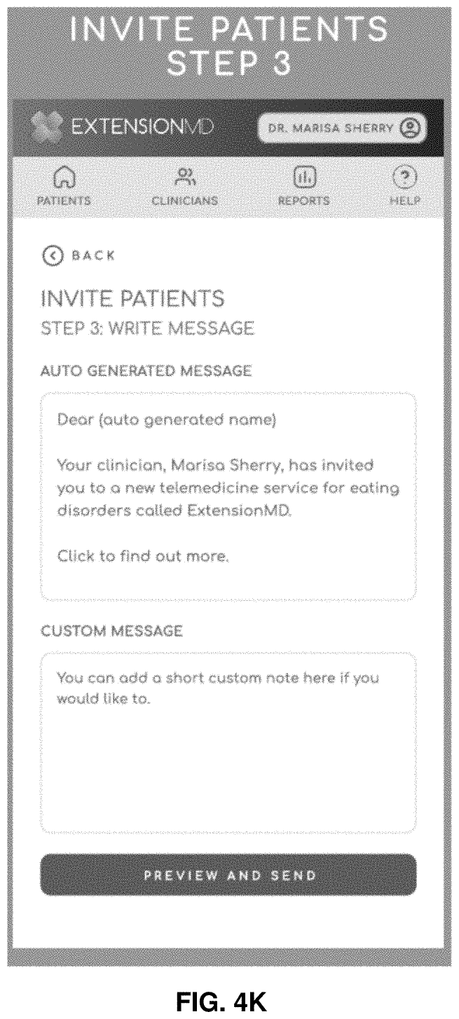

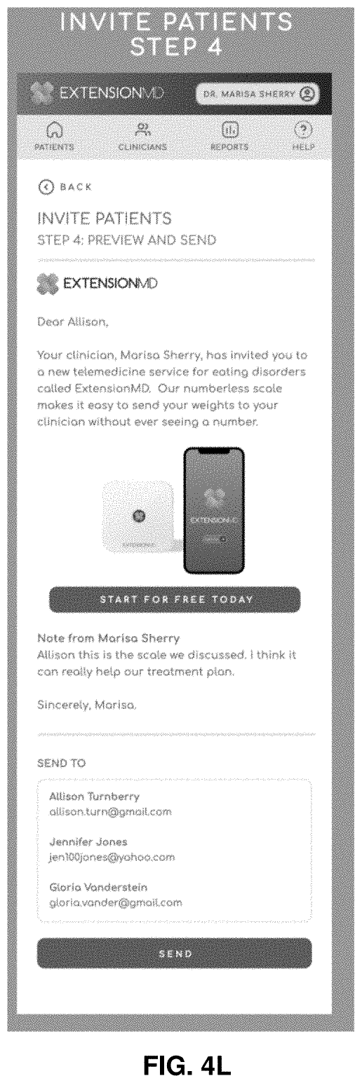

FIGS. 4A though 4L illustrate exemplary screenshots and an exemplary operation of the exemplary disclosed app.

In at least some exemplary embodiments, the exemplary disclosed system may include a healthcare module, comprising computer-executable code stored in non-volatile memory, a processor, a measurement device of a user, and a user device of the user. The healthcare module, the processor, the measurement device, and the user device may be configured to measure a bodily attribute data of the user using the measurement device, conceal the bodily attribute data from the user, transfer the bodily attribute data from the measurement device to the processor while concealing the bodily attribute data from the user, and provide output data based on the bodily attribute data to the user via the user device. The output data may exclude the bodily attribute data. The measurement device may be a bodyweight scale and the bodily attribute data may include a bodyweight of the user measured over time. The bodily attribute data includes at least one selected from the group of a bodyweight of the user, a Body Mass Index of the user, and combinations thereof. The exemplary disclosed system may also include a clinician device of a clinician, wherein the processor and the healthcare module provide the bodily attribute data, which is concealed from the user, to the clinician via the clinician device. The measurement device may be a numberless bodyweight scale. The healthcare module, the processor, the measurement device, and the user device may be configured to instruct the user to measure the bodily attribute data using the measurement device at random check-in times. The measurement device may be a bioelectric impedance analysis device that determines the bodily attribute data that may include body fat and body water levels of the user.

In at least some exemplary embodiments, the exemplary disclosed method may include providing a measurement device of a user, providing a user device of the user, providing a clinician device of a clinician, measuring a bodily attribute data of the user using the measurement device, concealing the bodily attribute data from the user, transferring the bodily attribute data from the measurement device to the clinician device while concealing the bodily attribute data from the user, and providing output data based on the bodily attribute data to the user via the user device.

The output data may exclude the bodily attribute data. The exemplary disclosed method may also include continuously preventing the user from directly receiving any information of the bodily attribute data. The exemplary disclosed method may further include providing alerts to the clinician via the clinician device of changes in the bodily attribute data, wherein the bodily attribute data includes the bodyweight of the user measured over time. The exemplary disclosed method may also include comparing changes in the bodily attribute data of the user measured over time with predetermined patterns of data associated with eating disorders. The exemplary disclosed method may further include prompting the user to enter input data via the user device, the input data including at least one selected from the group of food intake of the user during a period of time, exercise performed by the user during the period of time, sleep periods of the user during the period of time, self-assessed moods of the user during the period of time, and combinations thereof. A time of prompting and the input data requested in the prompting may be based on the bodily attribute data. The output data may include encouraging information for the user associated with the bodily attribute data that includes bodyweight data of the user.

The exemplary disclosed system, method, and apparatus may be used in any suitable healthcare application. For example, the exemplary disclosed system, method, and apparatus may be used in any suitable application for obtaining information remotely from patients without disclosing that information to patients. For example, the exemplary disclosed system, method, and apparatus may be used in any suitable application for obtaining weight information remotely from patients without disclosing that weight information to patients.

The exemplary disclosed system, method, and apparatus may provide an efficient and effective technique for efficiently and effectively treating patients who suffer from eating disorders. The exemplary disclosed system, method, and apparatus may provide a technique for efficiently obtaining weight information of a patient without the patient accessing that weight information. The exemplary disclosed system, method, and apparatus may provide accurate weight information using a remote mechanism.

An illustrative representation of a computing device appropriate for use with embodiments of the system of the present disclosure is shown in FIG. 5. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element 106, and one or more input/output devices/means 107, including one or more communication interfaces (e.g., RS232, Ethernet, Wifi, Bluetooth, USB). Useful examples include, but are not limited to, personal computers, smart phones, laptops, mobile computing devices, tablet PCs, and servers. Multiple computing devices can be operably linked to form a computer network in a manner as to distribute and share one or more resources, such as clustered computing devices and server banks/farms.

Various examples of such general-purpose multi-unit computer networks suitable for embodiments of the disclosure, their typical configuration and many standardized communication links are well known to one skilled in the art, as explained in more detail and illustrated by FIG. 6, which is discussed herein-below.

According to an exemplary embodiment of the present disclosure, data may be transferred to the system, stored by the system and/or transferred by the system to users of the system across local area networks (LANs) (e.g., office networks, home networks) or wide area networks (WANs) (e.g., the Internet). In accordance with the previous embodiment, the system may be comprised of numerous servers communicatively connected across one or more LANs and/or WANs. One of ordinary skill in the art would appreciate that there are numerous manners in which the system could be configured and embodiments of the present disclosure are contemplated for use with any configuration.

In general, the system and methods provided herein may be employed by a user of a computing device whether connected to a network or not. Similarly, some steps of the methods provided herein may be performed by components and modules of the system whether connected or not. While such components/modules are offline, and the data they generated will then be transmitted to the relevant other parts of the system once the offline component/module comes again online with the rest of the network (or a relevant part thereof). According to an embodiment of the present disclosure, some of the applications of the present disclosure may not be accessible when not connected to a network, however a user or a module/component of the system itself may be able to compose data offline from the remainder of the system that will be consumed by the system or its other components when the user/offline system component or module is later connected to the system network.

Referring to FIG. 6, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 6, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, iii) through a computing device 208, 209, 210 connected to a wireless access point 207 or iv) through a computing device 211 via a wireless connection (e.g., CDMA, GMS, 3G, 4G, 5G) to the WAN 201. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to.

The communications means of the system may be any means for communicating data, including image and video, over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

Turning now to FIG. 7, a continued schematic overview of a cloud-based system in accordance with an embodiment of the present invention is shown. In FIG. 7, the cloud-based system is shown as it may interact with users and other third party networks or APIs. For instance, a user of a mobile device 801 may be able to connect to application server 802. Application server 802 may be able to enhance or otherwise provide additional services to the user by requesting and receiving information from one or more of an external content provider API/website or other third party system 803, a constituent data service 804, one or more additional data services 805 or any combination thereof. Additionally, application server 802 may be able to enhance or otherwise provide additional services to an external content provider API/website or other third party system 803, a constituent data service 804, one or more additional data services 805 by providing information to those entities that is stored on a database that is connected to the application server 802. One of ordinary skill in the art would appreciate how accessing one or more third-party systems could augment the ability of the system described herein, and embodiments of the present invention are contemplated for use with any third-party system.

Traditionally, a computer program includes a finite sequence of computational instructions or program instructions. It will be appreciated that a programmable apparatus or computing device can receive such a computer program and, by processing the computational instructions thereof, produce a technical effect.

A programmable apparatus or computing device includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computing device can include any and all suitable combinations of at least one general purpose computer, special-purpose computer, programmable data processing apparatus, processor, processor architecture, and so on. It will be understood that a computing device can include a computer-readable storage medium and that this medium may be internal or external, removable and replaceable, or fixed. It will also be understood that a computing device can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the disclosure as claimed herein could include an optical computer, quantum computer, analog computer, or the like.

Regardless of the type of computer program or computing device involved, a computer program can be loaded onto a computing device to produce a particular machine that can perform any and all of the depicted functions. This particular machine (or networked configuration thereof) provides a technique for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Illustrative examples of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A data store may be comprised of one or more of a database, file storage system, relational data storage system or any other data system or structure configured to store data. The data store may be a relational database, working in conjunction with a relational database management system (RDBMS) for receiving, processing and storing data. A data store may comprise one or more databases for storing information related to the processing of moving information and estimate information as well one or more databases configured for storage and retrieval of moving information and estimate information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software components or modules, or as components or modules that employ external routines, code, services, and so forth, or any combination of these. All such implementations are within the scope of the present disclosure. In view of the foregoing, it will be appreciated that elements of the block diagrams and flowchart illustrations support combinations of means for performing the specified functions, combinations of steps for performing the specified functions, program instruction technique for performing the specified functions, and so on.

It will be appreciated that computer program instructions may include computer executable code. A variety of languages for expressing computer program instructions are possible, including without limitation C, C++, Java, JavaScript, assembly language, Lisp, HTML, Perl, and so on. Such languages may include assembly languages, hardware description languages, database programming languages, functional programming languages, imperative programming languages, and so on. In some embodiments, computer program instructions can be stored, compiled, or interpreted to run on a computing device, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computing device enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computing device can process these threads based on priority or any other order based on instructions provided in the program code.

Unless explicitly stated or otherwise clear from the context, the verbs “process” and “execute” are used interchangeably to indicate execute, process, interpret, compile, assemble, link, load, any and all combinations of the foregoing, or the like. Therefore, embodiments that process computer program instructions, computer-executable code, or the like can suitably act upon the instructions or code in any and all of the ways just described.

The functions and operations presented herein are not inherently related to any particular computing device or other apparatus. Various general-purpose systems may also be used with programs in accordance with the teachings herein, or it may prove convenient to construct more specialized apparatus to perform the required method steps. The required structure for a variety of these systems will be apparent to those of ordinary skill in the art, along with equivalent variations. In addition, embodiments of the disclosure are not described with reference to any particular programming language. It is appreciated that a variety of programming languages may be used to implement the present teachings as described herein, and any references to specific languages are provided for disclosure of enablement and best mode of embodiments of the disclosure. Embodiments of the disclosure are well suited to a wide variety of computer network systems over numerous topologies. Within this field, the configuration and management of large networks include storage devices and computing devices that are communicatively coupled to dissimilar computing and storage devices over a network, such as the Internet, also referred to as “web” or “world wide web”.

In at least some exemplary embodiments, the exemplary disclosed system may utilize sophisticated machine learning and/or artificial intelligence techniques to prepare and submit datasets and variables to cloud computing clusters and/or other analytical tools (e.g., predictive analytical tools) which may analyze such data using artificial intelligence neural networks. The exemplary disclosed system may for example include cloud computing clusters performing predictive analysis. For example, the exemplary neural network may include a plurality of input nodes that may be interconnected and/or networked with a plurality of additional and/or other processing nodes to determine a predicted result. Exemplary artificial intelligence processes may include filtering and processing datasets, processing to simplify datasets by statistically eliminating irrelevant, invariant or superfluous variables or creating new variables which are an amalgamation of a set of underlying variables, and/or processing for splitting datasets into train, test and validate datasets using at least a stratified sampling technique. The exemplary disclosed system may utilize prediction algorithms and approach that may include regression models, tree-based approaches, logistic regression, Bayesian methods, deep-learning and neural networks both as a stand-alone and on an ensemble basis, and final prediction may be based on the model/structure which delivers the highest degree of accuracy and stability as judged by implementation against the test and validate datasets.

Throughout this disclosure and elsewhere, block diagrams and flowchart illustrations depict methods, apparatuses (e.g., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “component”, “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present disclosure are contemplated for use with any language.

While multiple embodiments are disclosed, still other embodiments of the present disclosure will become apparent to those skilled in the art from this detailed description. There may be aspects of this disclosure that may be practiced without the implementation of some features as they are described. It should be understood that some details have not been described in detail in order to not unnecessarily obscure the focus of the disclosure. The disclosure is capable of myriad modifications in various obvious aspects, all without departing from the spirit and scope of the present disclosure. Accordingly, the drawings and descriptions are to be regarded as illustrative rather than restrictive in nature.

1. A system, comprising:
a healthcare module, comprising computer-executable code stored in non-volatile memory;
a processor;
a measurement device of a user; and
a user device of the user;
wherein the healthcare module, the processor, the measurement device, and the user device are configured to:
measure a bodily attribute data of the user using the measurement device;
conceal the bodily attribute data from the user;
transfer the bodily attribute data from the measurement device to the processor while concealing the bodily attribute data from the user; and
provide output data based on the bodily attribute data to the user via the user device;
wherein the output data excludes the bodily attribute data.

2. The system of claim 1, wherein the measurement device is a bodyweight scale and the bodily attribute data includes a bodyweight of the user measured over time.

3. The system of claim 1, wherein the bodily attribute data includes at least one selected from the group of a bodyweight of the user, a Body Mass Index of the user, and combinations thereof.

4. The system of claim 1, further comprising a clinician device of a clinician, wherein the processor and the healthcare module provide the bodily attribute data, which is concealed from the user, to the clinician via the clinician device.

5. The system of claim 1, wherein the measurement device is a numberless bodyweight scale.

6. The system of claim 1, wherein the healthcare module, the processor, the measurement device, and the user device are configured to instruct the user to measure the bodily attribute data using the measurement device at random check-in times.

7. The system of claim 1, wherein the measurement device is a bioelectric impedance analysis device that determines the bodily attribute data that includes body fat and body water levels of the user.

8. A method, comprising:
providing a measurement device of a user;
providing a user device of the user;
providing a clinician device of a clinician;
measuring a bodily attribute data of the user using the measurement device;
concealing the bodily attribute data from the user;
transferring the bodily attribute data from the measurement device to the clinician device while concealing the bodily attribute data from the user; and
providing output data based on the bodily attribute data to the user via the user device;
wherein the output data excludes the bodily attribute data.

9. The method of claim 8, further comprising continuously preventing the user from directly receiving any information of the bodily attribute data.

10. The method of claim 8, further comprising providing alerts to the clinician via the clinician device of changes in the bodily attribute data, wherein the bodily attribute data includes the bodyweight of the user measured over time.

11. The method of claim 8, further comprising comparing changes in the bodily attribute data of the user measured over time with predetermined patterns of data associated with eating disorders.

12. The method of claim 8, further comprising prompting the user to enter input data via the user device, the input data including at least one selected from the group of food intake of the user during a period of time, exercise performed by the user during the period of time, sleep periods of the user during the period of time, self-assessed moods of the user during the period of time, and combinations thereof.

13. The method of claim 12, wherein a time of prompting and the input data requested in the prompting are based on the bodily attribute data.

14. The method of claim 8, wherein the output data includes encouraging information for the user associated with the bodily attribute data that includes bodyweight data of the user.

15. The method of claim 8, further comprising transmitting a check-in data from the clinician device to the user device, the check-in data instructing the user to measure the bodily attribute data using the measurement device.

16. The method of claim 8, further comprising determining a propensity score based on the bodily attribute data, the propensity score indicating a propensity of the user to binge eat or to purge following eating.

17. A system, comprising:
a healthcare module, comprising computer-executable code stored in non-volatile memory;
a processor;
a numberless bodyweight scale of a user; and
a user device of the user;
wherein the healthcare module, the processor, the numberless bodyweight scale, and the user device are configured to:
measure a bodyweight data of the user using the numberless bodyweight scale;
conceal the bodyweight data from the user;
transfer the bodyweight data from the numberless bodyweight scale to the processor while concealing the bodyweight data from the user; and
provide output data based on the bodyweight data to the user via the user device;
wherein the output data excludes the bodyweight data.

18. The system of claim 17, further comprising a clinician device of a clinician, wherein the processor and the healthcare module provide the bodyweight data, which is concealed from the user, to the clinician via the clinician device.

19. The system of claim 17, wherein the healthcare module, the processor, the numberless bodyweight scale, and the user device are configured to instruct the user to measure the bodyweight data using the numberless bodyweight scale at random check-in times.

20. The system of claim 17, wherein the numberless bodyweight scale further includes a bioelectric impedance analysis device that determines body fat and body water levels of the user.
